(19) United States
(12) Patent Application Publication
(10) Pub. No.: US 2023/0270420 A1
(43) Pub. Date: Aug. 31, 2023

(54) URINE SAMPLE ANALYSIS SYSTEM

(71) Applicant: ORUBA MEDIKAL TEKNOLOJI ARGE IC VE DIS TIC. LTD. STI, Ankara (TR)

(72) Inventors: Ahmet ULUDAG, Ankara (TR); Ali Ogulcan DULGER, Ankara (TR); Utku ULUSAHIN, Ankara (TR)

(73) Assignee: ORUBA MEDIKAL TEKNOLOJI ARGE IC VE DIS TIC. LTD. STI, Ankara (TR)

(21) Appl. No.: 18/016,364

(22) PCT Filed: Jul. 16, 2021
(86) PCT No.: PCT/TR2021/050736

§ 371 Date: Jan. 16, 2023

(30) Foreign Application Priority Data
Jul. 16, 2020 (TR) 2020/11323
Jul. 15, 2021 (TR) 2021/011575

(51) Int. Cl. A61B 10/00; G01N 33/493; B01L 3/00; A61F 5/451

(52) U.S. Cl. CPC A61B 10/007; A61F 5/451; B01L 3/502707; G01N 33/493; B01L 2300/0825

(57) ABSTRACT

A urine specimen analysis system that allows the chemical content of a urine specimen to be analysed via camera and test strips in order for the current state of health of persons to be understood is disclosed. A urine analysis system that carries out the analysis on the test strip using a required amount of urine specimen is disclosed. A urine analysis system that will ensure the chemical analysis of the urine by observing it with a camera after waiting for a period of time during which the urine strip will realise its chemical reactions. With its feature to clean itself and the test strips, the urine analysis system will be able to prevent mixing of both the previous and next users’ urines by cleaning the urine line and the system.

### CROSS REFERENCE TO THE RELATED APPLICATIONS

This application is the national stage entry of International Application No. PCT/TR2021/050736, filed on Jul. 16, 2021, which is based on and claims priority to Turkish Patent Application Nos. 2020/11323, filed Jul. 16, 2020 and 2021/011575, filed Jul. 15, 2021, the entire contents of which are incorporated herein by reference.

### TECHNICAL FIELD

This invention relates to a urine specimen analysis system that allows the chemical content to be analysed via camera and test strips in order for the current state of health of persons to be understood from their urine specimens.

### BACKGROUND

By performing content analysis of urine specimens, it is possible to diagnose many diseases or to diagnose the instant state of health. Absorbent surface to which reactive chemicals had been absorbed are used for the detection of chemical content. These absorbent surfaces change colours when the chemicals they contain react with the urine. By means of this colour change, the amounts of certain chemicals within the urine can be detected by the help of a scale.

The biomarkers the amount of which can be detected using the test strip in the urine are; proteins, glucose, ketone, haemoglobin, bilirubin, urobilinogen, acetone, nitrite and leukocytes. These biomarkers provide data to physicians so that they can diagnose many diseases. Therefore, tests on urine specimens are very often made in health institutions using the content analysis strips.

Today, there are patent applications related to automatic urine tests by means of strips placed inside toilets. One of these is described in United States patent applications no US1060571. In the said patent application, carrying out a urine analysis from the urine of the user by means of a plurality of strips placed under a water closer.

With mobile phones becoming widespread, carrying out this analysis anywhere with the help of a urine test strip and a mobile phone having a camera has become possible. One example of this is described in the patent application CN105223196. The camera of the mobile device detects the amount of biomarkers within the urine from the colour changes on the strip through an application, informs the user and can also save this information.

### SUMMARY

This invention aims to realise a urine analysis system that carries on to the test strip in an amount that is required for the urine specimen to be analysed using the test strip. Another aim of the invention is to develop a urine analysis system that will ensure the chemical analysis of the urine by observing it with a camera after waiting for a period of time during which the urine strip will realise its chemical reactions. With its feature to clean itself and the test strips, the urine analysis system will be able to prevent both the previous and next users’ urines to mix by means of cleaning the urine line and the system to stink.

The urine analysis system realised for the invention to reach its aims was shown in the attached figures.

### BRIEF DESCRIPTION OF THE DRAWINGS

FIG. 1 - Front view of the urinal embodiment of the urine specimen analysis system.

FIG. 2 - Rear view of the urinal embodiment of the urine specimen analysis system.

FIG. 3 - Perspective view of the internal parts of the urinal or water closet of the urine specimen analysis system.

FIG. 4 - Perspective view of the internal parts of the urinal or water closet of the urine specimen analysis system from the cassette cover side.

FIG. 5 - Perspective view of the internal parts the urinal or water closet of the urine specimen analysis system with the cassette cover open and cassette out.

FIG. 6 - Perspective view of the internal parts of the urinal or water closet of the urine specimen analysis system showing where the needle detail is taken.

FIG. 7 - Perspective view of the internal parts of the urinal or water closet of the urine specimen analysis system showing the needle detail.

FIG. 8 - View of an exemplary part of the test strip used in the urine specimen analysis system.

FIG. 9 - B-B sectional view of the urine specimen analysis system mounted within the urinal.

FIG. 10 - Front view showing the axis where the B-B sectional view of the urine specimen analysis system mounted within the urinal is taken.

FIG. 11 - Schematic view with a sectional line taken from the middle of the urine specimen analysis system mounted within the water closet.

FIG. 12 - Schematic perspective view of the urine specimen analysis system including needle and vacuum line.

FIG. 13 - Detailed schematic perspective view of the urine specimen analysis system including the needle and vacuum line.

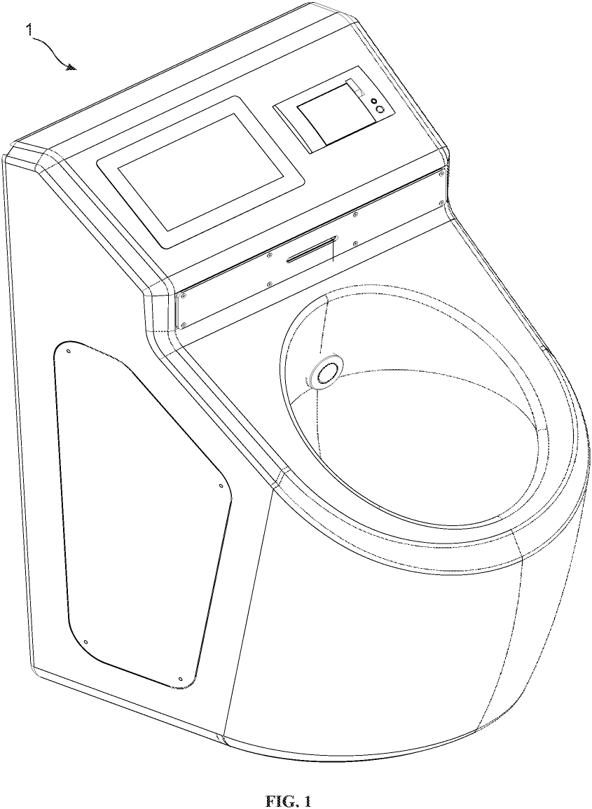

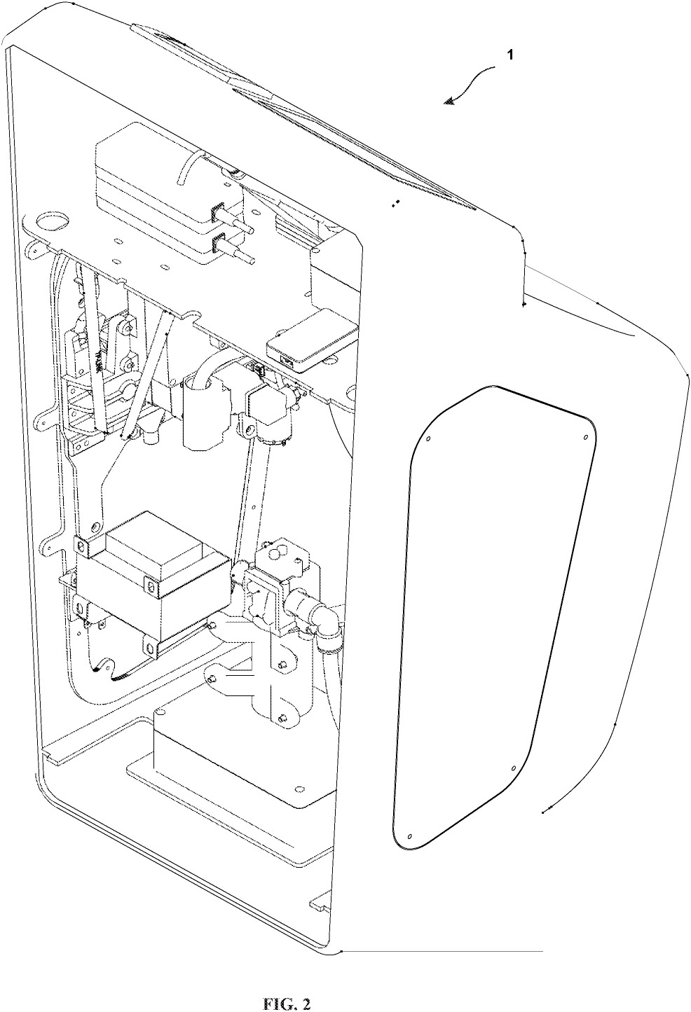

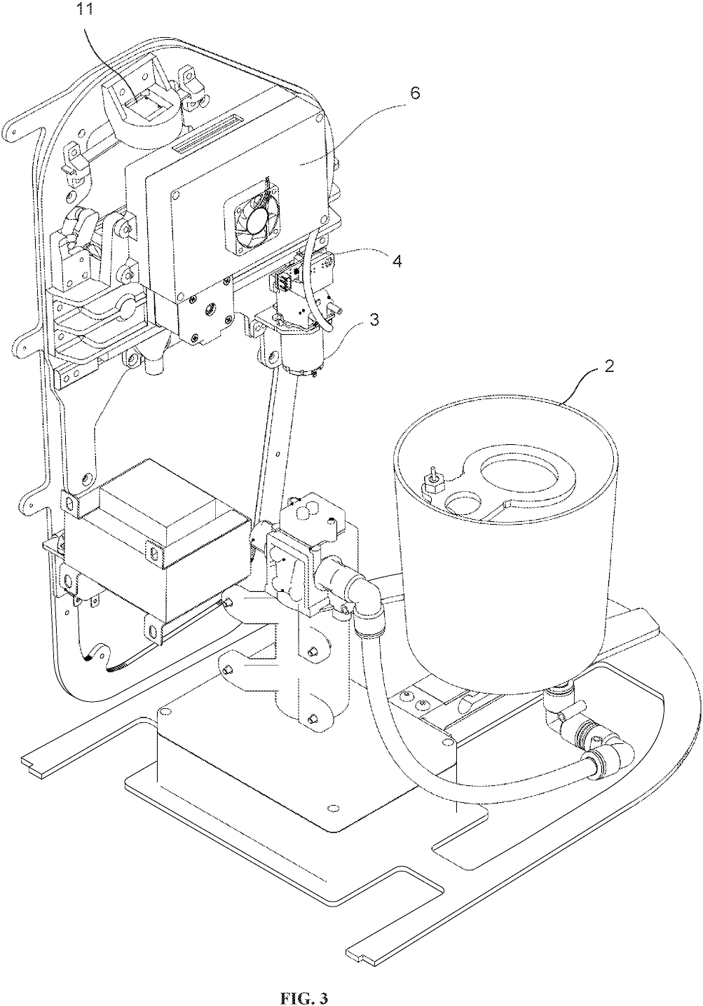

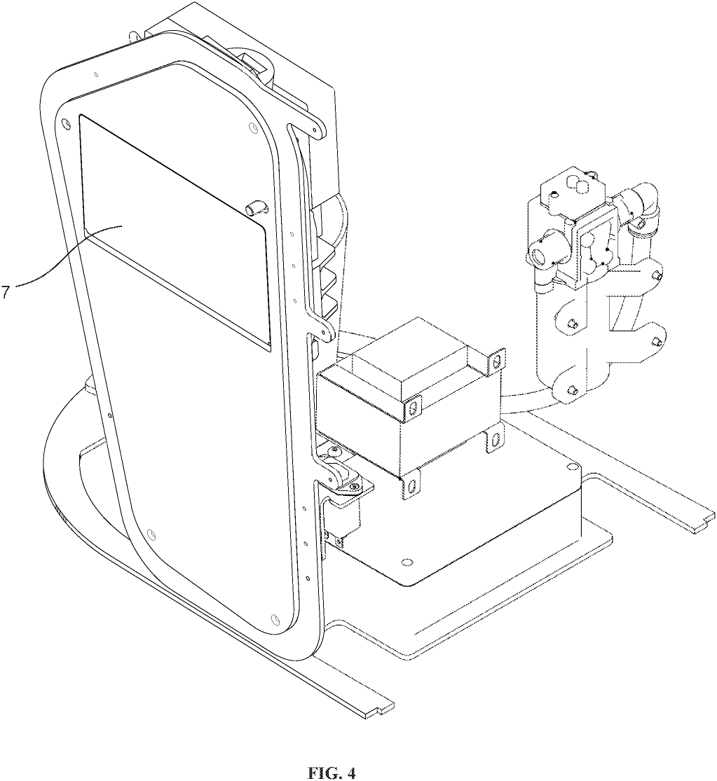

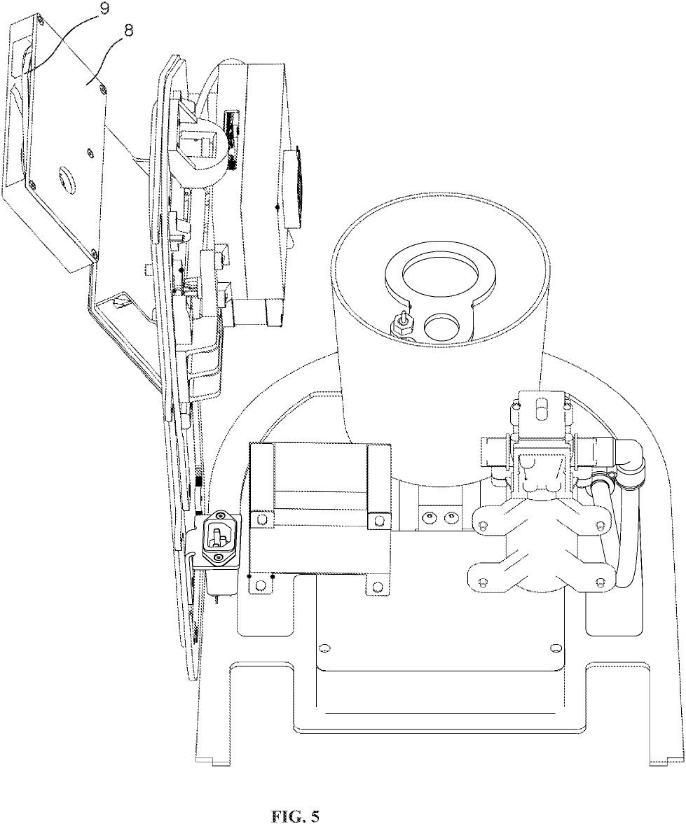

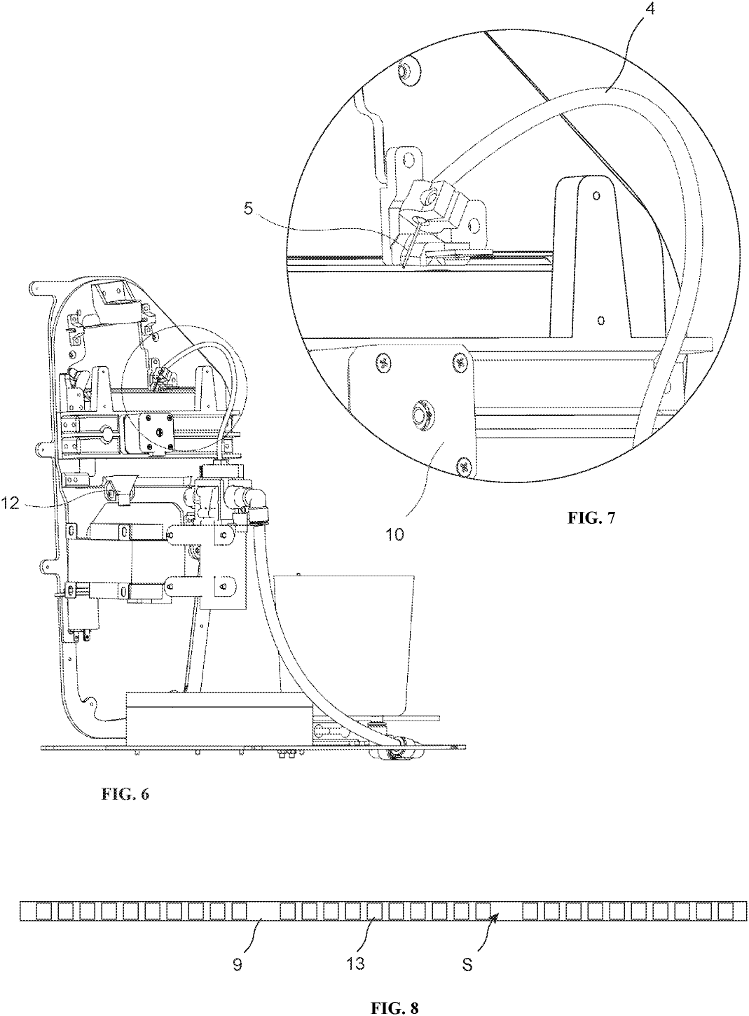

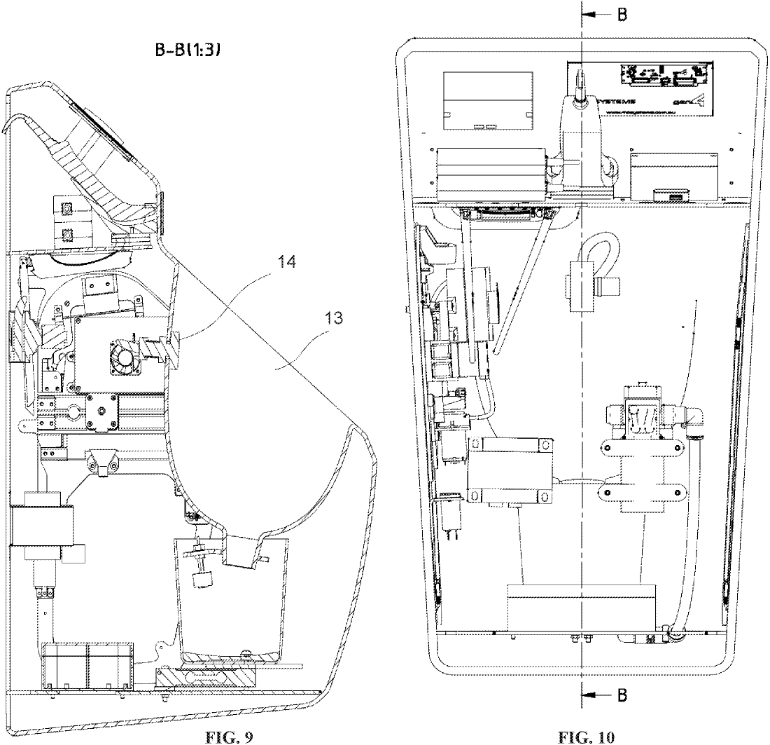

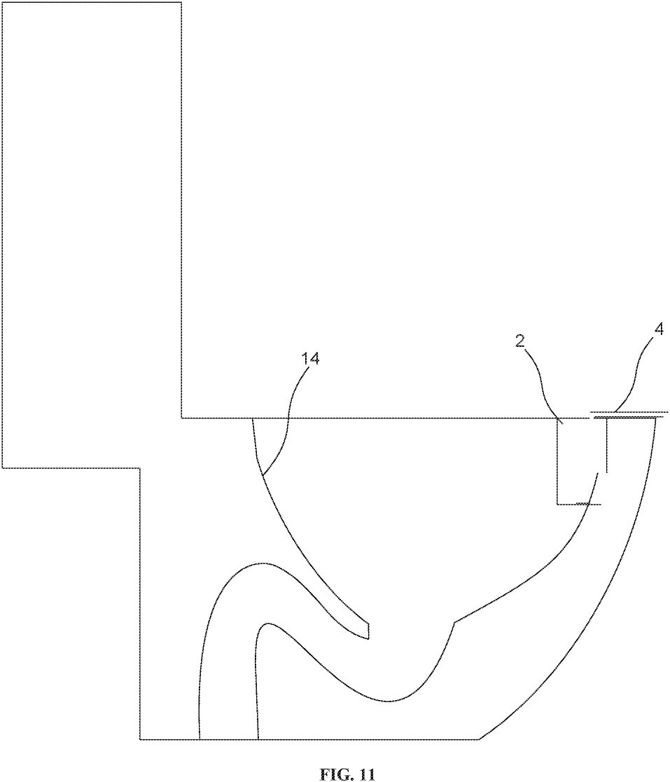

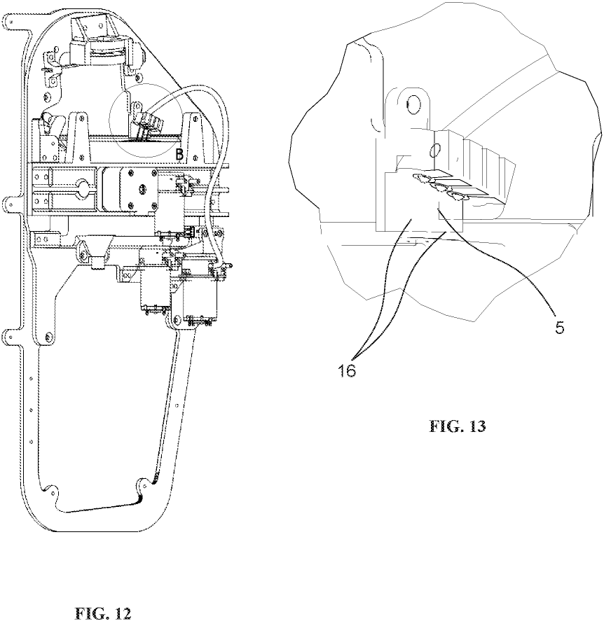

The parts in the figures are enumerated and the parts the numbers refer to are given below.
1. Urine Specimen Analysis System
2. Collection container
3. Engine
4. Urine feed line
5. Needle
6. Control unit
7. Cassette cover
8. Cassette
9. Test strip
10. Cassette motor
11. Camera
12. Urine collection funnel
13. Absorbent layer
14. Bowl
15. Water outlet
16. Vacuum line

### DETAILED DESCRIPTION OF THE EMBODIMENTS

Urine specimen analysis system (1) that is the subject of the invention, in its simplest form, includes a water closet or a urinal bowl/body into which urine specimen can be given, test strip (9), camera (11) and a pump engine (3) and needle (5) that carry the urine to the test strip (9). The needle (5) ensures that urine is carried on to the test strip (9) directly in sufficient amount.

By the help of the urine reaching on the absorbent layers (13) on the test strip (9) sufficient enough for the reaction, the colour change on the layers will be observed. Additionally, as sufficient amount of urine will be taken from the needle (5) the amount of urine that will stink in the system will be reduced.

The test strip (9) shall move linearly in front of the needle (5) for the test to be performed. The speed and amount of the movement of the test strip (9) varies depending on the total number of layers (13) on the test strip (9) onto which the test chemical was absorbed. While it is enough for the test strip (9) to move as long as the urine absorbance region to perform 3 tests in front of the needle (5) in the case of testing for 3 different chemicals, the amount of movement will cover regions for 10 test on a test strip (9) comprising 10 tests. In addition to that, to prevent each urine flow from polluting the next test region on the test strip (9), there is a certain spacing (S) between each test strip (9) section.

The process of advancing the test strip (9) as far as all the test absorbent layer (13) surfaces and the spacing itself is carried out by a cassette motor (10). Because while the test strip (9) is wrapped in a cassette (8), the urine sample can be inserted in and removed from the urine specimen analysis system (1). The cassette (8) is the most suitable form for storing, reading, writing of a strip material and moving any strip inside it. For this reason, a cassette cover (7) is attached to the body of the urine specimen analysis system (1) and the cassette (8) can be attached and removed by means of it. The cassette motor (10), which will enable the test strip (9) to be rotated by attaching the cassette (8) in place, enters into the slot where it will move the reel of the cassette (8).

The amount of movement and speed of the cassette motor (10) will be determined according to the operational speed of the pump motor (3) and the amount of urine coming from the tip of the needle (5). If the cassette motor (10) moves fast, enough urine may not reach the absorbent layers (13). In the contrary case, it is possible that the excess urine will fill into the cassette (8) and pollute it in a way to stink.

The method of embodiment of the urine specimen analysis system (1), that is the subject of the invention, shown in the attached figures, includes a component group that can be mounted inside a urinal or a water closet or on the wall. The most important element in this component group is the collection container (2) in which the urine is collected. The collection container (2) is placed under the bowl (14) for the urinal embodiment shown in the figures.

In water closet bowl type embodiments, a collection container (2) can be placed close to the front of the closet, not under it, to collect the urine specimen so that solid faeces does not mix with the urine. It is possible for a urine feed line (4) coming out of this collection container (2) to suck the urine with the suction of the pump motor (3) and deliver it to the test strip (9).

Whether in urinal or water closet bowl embodiment, the whole process will be carried out by a control unit (6). Entire process including telling the users that the system is ready for the test and they can start urinating, and the process of sucking the urine with the feed line (4) when it is collected in the collection container (2) and delivering it to the test strip (9) will be controlled by the control unit (6). The chemicals contained in the absorbent layers (13) change colour over a period of time when they encounter urine. This time varies between 30 seconds and 120 seconds depending on the chemical entering the reaction. During this period, the waiting and observing the amount of change with the camera (11) is also tracked by the control unit (6).

Cleaning the test strip (9) and absorbent layers (13) after each test run is a necessity to avoid stinking. For this purpose, own water outlet (15) system of the urinal or water closet will be used. Generally, the high pressure water provided by this water outlet (15) can ensure the cleaning of the entire surface of the bowl (14) to which the urine had contacted. In this way, by means of the water that can also reach the collection container (2), it is possible to supply water to the entire urine feed line (4) and to deliver this water up to the test strip (9). The test strip (9) will be washed with a large amount of water and will be purified from urine residues.

At the end of each process, first of all, the needle (5) and the feed line (4), which are emptied by vacuum instead of spraying, will be cleaned. Discharging of the water sent on the test strip (9) and the urine residues after that is important for the stable operation of the system. For this reason, there is an opening under the cassette (8) for the discharging of the urine and water filled into the cassette (8). Water and urine residues coming off this opening are collected by the urine collection funnel (12) and sent to the drain.

The total number of tests inside the test strip (9), the length of the strip, the length and number of the separating partitions (S) separating the absorbent layer (13) groups from one another are predefined in the control unit (6). In addition to that, the visual data provided by the camera (11) and the number of rotation of the cassette motor (10) will be used in the continuous control and calibration of these values.

The amount of urine delivered onto the test strip (9) by means of the needle (5) may be higher than the amount of absorption of the absorbent layers (13), and it is possible for urine droplets to remain on the test strip (9) apart from the absorbent layers (13). These urine droplets both cause stinking and the images taken by the camera (11) to deteriorate. Urine droplets should be collected from over the test strip (9) as a precaution against both problems.

On the other hand, it is possible for the urine coming from the needle (5) to flow in large amounts and suddenly due to a problem similar to the sudden clearance of an obstruction. In this case, to prevent the pollution of the test strips (9) that are kept as clean rolls, the excess urine should be extracted not to contaminate the other strips.

The vacuum line (16) will be used to collect excess urine from the test strip (9) area on which the needle (5) drips urine. The vacuum line (16) will ensure that the excess urine that the absorbent layers (13) do not absorb will be removed from the front, back, right, left and even from the entire circumference of the needle (5) angularly by means of one or more vacuum pumps. The proximity of the vacuum line (16) to the needle (5), the vacuum power, the frequency and sequence of operation can be adjusted to ensure that no urine drops are left on the test strip (9).

For the camera (11) to easily detect colour changes on the test strip (9), a printed image containing reference colours can be placed in a region within the camera’s (11) field of view. By this way, as the ambient lighting will illuminate both the test strip (9) and the printed images at the same time, the measurement will be made without being affected by the wavelength changes.

In a different embodiment of the urine specimen analysis system (1) that is the subject of the invention, instead of taking the urine to be used to treat the absorbent layers (13) with urine to perform the analysis from the collection container (2) via the urine feed line (4), it is possible to feed it by means of a feed line (4) from the urine accumulating in the bowl (14) during urination or from a drain line directed from the bowl (14). In this embodiment of the invention, although there is no accumulation of urine in a reservoir, it will be possible to collect urine from any line during the transfer of urine from the bowl (14) to the drain.

In another embodiment of the urine specimen analysis system (1) that is the subject of the invention, it is possible that the test strip (9) to be used for urinalysis not to be wrapped in a cassette (8). It is possible for the test strip layout, which can be designed in different geometric shapes, to be placed in a closed field. In this case, the thing that is tried to be explained is to include test strips (9) to be used in a closed environment that is desired to be told with the use of cassettes (8).

Again, the camera (11) positioned to face the test strip (9) directly or with different optical means and is to be used in the urine specimen analysis system (1) that is also the subject of the invention, can be used as a colour sensor or image sensor or wavelength sensor.

In water closet type embodiments, it is possible to use a separator/sorter that separates solid and liquid faeces from each other instead of the bowl (14). By means of the separator/sorter used during this embodiment, it is possible to ensure that the urine remains in the front of the separator in accordance with human ergonomics, and direct the solid faeces to the rear side in accordance with the same female and male ergonomics. Due to the nature of body ergonomics of a man or woman in a sitting position is expected to urinate forward and defecate backwards/to the ground. Accordingly, the separator/sorter that separates the incoming faeces into two parts, will be able to direct urine and faeces to different places with its two funnel structures.

What is claimed is:

1. A urine specimen analysis system to understand a current state of health of persons from a chemical content of a urine specimen, comprising:
a test strip within a cassette,
a camera facing the test strip,
a needle delivering the urine specimen to the test strip; wherein
a urine feed line directs the urine specimen to the test strip from an accumulation in a bowlor from a drain line directed from the bowlfor the urine specimen to be carried to an absorbent layer of the test strip.

2. The urine specimen analysis systemof claim 1, wherein a vacuum is used instead of a spray to clean the urine specimen remaining in the needleand the urine feed line.

3. The urine specimen analysis systemof claim 1, wherein a discharging of remains of the urine specimen is done by sending water on to the test strip.

4. The urine specimen analysis systemof claim 1, wherein the remains of the urine specimen drop out through a urine collection funnel from an opening under the cassette for a discharging of the urine specimen and filling of water into the cassetteis collected and discharged.

5. The urine specimen analysis systemof claim 1, wherein a vacuum line ensures that an excess urine not absorbed by the absorbent layer is removed froma front, a back, a right, a left and from an entire circumference of the needle angularly by means of one or more vacuum pumps, wherein an excess of the urine specimen from an area of the test strip where the needle drips the urine specimen is collected.

6. The urine specimen analysis systemof claim 1, wherein a collection container collects the urine specimen from an area close to a front side of a water closet so that a solid feces does not mix with the urine specimen in the water closet.

7. The urine specimen analysis systemof claim 1, wherein a separator/sorter ensures a separation of a solid feces and a liquid feces in a water closet and the separator/sorter directs the liquid feces and the solid faces to different directions by means of two funnel structures; wherein during urination of a man or a woman, the liquid feces is directed to a front direction and during defecation, the solid feces is directed to a backward direction or to the ground when the man or the woman is in a sitting position.

8. A urine specimen analysis system to understand a current state of health of persons from a chemical content of a urine specimen, comprising:
a test stripwithin a cassette,
a camera facing the test strip,
a needle deliver[ing the urine specimen to the test strip; wherein
a cassette motor moves the test strip in front of the needle and a collection container is placed inside or under a bowl, wherein the urine specimen reaches the collection container and a feed line sucks out the urine specimen from inside of the collection container.

9. The urine specimen analysis systemof claim 8 wherein a vacuum instead of a spray is used to clean the urine specimen remaining in the needleand the feed line.

10. The urine specimen analysis systemof claim 8, wherein a discharge of water and remains of the urine specimen is done by sending on the test strips.

11. The urine specimen analysis systemof claim 8, wherein remains of the urine specimen drop out through a urine collection funnel from an opening under the cassettefor a discharging of the urine specimen and filling of water into the cassette is collected and discharged.

12. The urine specimen analysis systemof claim 8, wherein a vacuum line ensures an excess of the urine specimen not absorbed by an absorbent layer is removed from a front, a back, a right, a left and from an entire circumference of the needleangularly by means of one or more vacuum pumps, wherein the excess of the urine specimen is collected from an area of the test strip where the needle drips the urine specimen.

13. The urine specimen analysis system of claim 8, wherein a collection container collects the urine specimen from an area close to a front side of a water closet so that a solid feces does not mix with the urine specimen in the water closet.

14. The urine specimen analysis systemof claim 8, wherein a separator/sorter ensures a separation of a solid feces and a liquid feces in a water closet and wherein the separator/sorter directs the liquid feces and the solid feces to different directions by means of two funnel structures; wherein during urination of a man or a woman, the liquid feces is directed to a front direction and during defecation, the solid feces is directed to a backward direction or to the ground when the man or the woman is in a sitting position.

15. The urine specimen analysis system of claim 2, wherein a discharging of remains of the urine specimen is done by sending water onto the test strip.

16. The urine specimen analysis system of claim 2, wherein the remains of the urine specimen drop out through a urine collection funnel from an opening under the cassette for a discharging of the urine specimen and filling of water into the cassette is collected and discharged.

17. The urine specimen analysis system of claim 3, wherein the remains of the urine specimen drop out through a urine collection funnel from an opening under the cassette for a discharging of the urine specimen and filling of water into the cassette is collected and discharged.

18. The urine specimen analysis system of claim 2, wherein a vacuum line ensures that an excess urine not absorbed by the absorbent layer is removed from a front, a back, a right, a left and from an entire circumference of the needle angularly by means of one or more vacuum pumps, wherein an excess of the urine specimen from an area of the test strip where the needle drips the urine specimen is collected.

19. The urine specimen analysis system of claim 3, wherein a vacuum line ensures that an excess urine not absorbed by the absorbent layer is removed from a front, a back, a right, a left and from an entire circumference of the needle angularly by means of one or more vacuum pumps, wherein an excess of the urine specimen from an area of the test strip where the needle drips the urine specimen is collected.

20. The urine specimen analysis system of claim 4, wherein a vacuum line ensures that an excess urine not absorbed by the absorbent layer is removed from a front, a back, a right, a left and from an entire circumference of the needle angularly by means of one or more vacuum pumps, wherein an excess of the urine specimen from an area of the test strip where the needle drips the urine specimen is collected.
